(19) United States
(12) Patent Application Publication
(10) Pub. No.: US 2022/0381755 A1
(43) Pub. Date: Dec. 1, 2022

(54) SYNCHRONOUS SAMPLING AND MEASURING SYSTEM AND METHOD THEREOF FOR FLUE GAS PARTITION

(71) Applicant: HUANENG POWER INTERNATIONAL, INC. SHANGHAI SHIDONGKOU FIRST POWER PLANT, Shanghai (CN)

(72) Inventors: Hao MAO, Shanghai (CN); Feng HAN, Shanghai (CN); Lingling XIAO, Shanghai (CN); Yudong SHI, Shanghai (CN); Ying GUAN, Shanghai (CN); Yuhao ZHANG, Shanghai (CN); Xuan ZHAO, Shanghai (CN); Jiaying ZHU, Shanghai (CN)

(21) Appl. No.: 17/447,434

(22) Filed: Sep. 12, 2021

(30) Foreign Application Priority Data
May 28, 2021 (CN) 202110589013.7

(51) Int. Cl. G01N 33/00; G01P 5/00; F23J 15/02

(52) U.S. Cl. CPC G01N 33/0027; G01P 5/00; F23J 15/02; F23J 2215/10

(57) ABSTRACT

The present invention relates to a synchronous sampling and measuring system and a method thereof for flue gas partition. The system includes a detection device arranged at an SCR outlet for simultaneous detection of flue gas from the pipelines in different areas, wherein the detection device is connected to a speed measurement device for measuring the speed of flue gas, the speed measurement device is connected to a central control unit and one end of a valve group, respectively, the other end of the valve group is connected to an air extracting device and a dilution unit, respectively, the control end of the valve group and the control end of the air extracting device are connected to a central control unit, respectively, the dilution unit is connected to a CEMS analyzer.

### CROSS-REFERENCE TO RELATED APPLICATIONS

The present application claims priority to Chinese Patent Application No. 202110589013.7 filed on May 28, 2021, which is incorporated herein by reference in its entirety.

### TECHNICAL FIELD

The present invention relates to the technical field of denitrification monitoring in thermal power plants, in particular to a synchronous sampling and measuring system and a method thereof for flue gas partition.

### BACKGROUND ART

At present, an SCR denitration process is used for denitration in most of the units in thermal power plants, for most of the existing conventional SCR system, NOₓ and O₂ monitoring analyzers are usually set at the SCR inlet and outlet, an NH₃ monitoring analyzer is arranged at the SCR outlet, the amount of injected ammonia is controlled by the measured value of the NOₓ and O₂ monitoring analyzers at the SCR inlet and outlet, the measured value of the NH₃ monitoring analyzer at the SCR outlet, and the flue gas flow. With the all-round promotion of the ultra-low emission standard, the SCR denitration efficiency of the unit often exceeds 90%, so most units adopt the measurement control method for partition, i.e., a new set of NOₓ sampling and measuring device for partition is added at the outlet of the SCR area to measure the NOₓ in the SCR area in partition, so as to monitor in real time the denitration operation of the SCR area and partitioning reaction of the catalyst, and the amount of catalyst sprayed in partition can be dynamically adjusted according to the NOₓ distribution at the SCR outlet, to avoid the problem of large deviations in NOₓ concentration distribution.

The traditional NOₓ sampling and measuring system for partition at an SCR outlet is shown in FIG. 1, wherein each sampling probe is directly connected with each dilution unit, i.e., the dilution unit corresponds to the sampling probe one by one, and the dilution unit dilutes the flue gas taken out by the sampling probe, then by controlling the evacuation volume of the evacuating device and adjusting the length of the pipeline between the sample gas and the analyzer, and in combination with the reversing mechanism to switch the pipeline, the flue gas from different sampling points at the same time can be transported to the CEMS analyzer at different time in an orderly manner, thereby realizing the object of real-time measurement for partition.

However, the above structure has the following shortcomings:

(1) Each probe has an independent dilution unit, easily leading to measurement errors of different sampling units.

Different measurement units are configured with different dilution units, and the dilution units are internally composed of small sonic orifices. The difference in the accuracy of the orifices has a great highly sensitive effect on the dilution ratio, and it is difficult to ensure the same dilution ratio of each measurement unit. Normally, the error of dilution ratio can be compensated by the CEMS analyzer at the back end, but usually the analyzer corresponds to the dilution unit one by one, when a plurality of dilution units correspond to one gas analyzer, the measurement error caused by the difference of dilution ratio between dilution units cannot be compensated, and then the measurement accuracy is affected.

(2) The difference in gas flow rate after dilution by the dilution unit easily leads to real-time difference during measurement.

By controlling the evacuation volume of the evacuating device and adjusting the length of the pipeline between the sample gas and the analyzer, although the flue gas passing through different sampling points at the same time can be transported to the analyzer at different time in an orderly manner, the adjusted length of the pipeline is fixed after the installation of the equipment, and the diluted flue gas pressure is low and easily affected by the bending degree of the pipeline and attachments of the pipeline, etc., thereby leading to a great difference between the time at which the analyzed flue gas reaches the analyzer and the designed time, and further producing real-time difference during measurement.

### SUMMARY OF THE INVENTION

The object of the present invention is to overcome the above shortcomings existing in the prior art and provide a synchronous sampling and measuring system and method thereof for flue gas partition, to improve measurement precision and reduce real-time difference during measurement.

The object of the present invention is realized through the following technical solutions: a synchronous sampling and measuring system for flue gas partition includes a detection device arranged at an SCR outlet for simultaneous detection of flue gas from the pipelines in different areas, wherein the detection device is connected to a speed measurement device for measuring the speed of flue gas, the speed measurement device is connected to a central control unit and one end of a valve group, respectively, the other end of the valve group is connected to the air extracting device and the dilution unit, respectively, the control end of the valve group and the control end of the air extracting device are connected to the central control unit, respectively, the dilution unit is connected to a CEMS analyzer, the central control unit correspondingly controls the on and off of the valve group and controls the working state of the air extracting device according to the data output from the speed measurement device, such that the flue gas sampled at the same time in the flue is transported to the air extracting device according to the set time sequence.

Further, the dilution unit is fed with zero gas.

Further, an outlet of the air extracting device is communicated with a furnace.

Further, a calibration device for calibrating and correcting the working state and precision of the speed measurement device, the valve group, the air extracting device and the dilution unit is connected between the detection device and the speed measurement device.

Further, the calibration device is fed with standard gas.

Further, the detection device includes a plurality of probes.

Further, the speed measurement device includes a plurality of speed measurement units corresponding to different probes.

Further, the plurality of speed measurement units are respectively connected to the central control unit.

Further, the valve group includes a plurality of valve switches corresponding to different speed measurement units.

Further, the calibration device includes a plurality of calibration units corresponding to different probes.

A synchronous sampling and measuring method for flue gas partition includes the following steps:

S1, simultaneously acquiring, by the detection device, flue gas from pipelines in different areas at the SCR outlet;

S2, measuring, by the speed measurement device, the speed of the flue gas from pipelines in different areas acquired by the detection device, and outputting the speed measurement value of the flue gas from pipelines in different areas to the central control unit;

S3, correspondingly controlling, by the central control unit, the on and off of the valve group and controlling the working state of the air extracting device according to the speed measurement value of the flue gas from pipelines in each area and in combination with the set control requirements, such that the flue gas sampled at the same time in different pipelines enters the air extracting device according to the set time sequence;

if the valve group correspondingly conducts a certain pipeline to the passage of the air extracting device, then the air extracting device extracts the flue gas in the pipeline, so that the flue gas in the pipeline is diluted through the dilution unit, and then transported to the CEMS analyzer for flue gas analysis, to obtain the flue gas measurement results corresponding to the pipeline; and

S4, outputting, by the CEMS analyzer, the flue gas measurement results corresponding to pipelines in different areas, that is, the synchronous sampling and measuring process for flue gas partition is completed.

Compared with the prior art, the present invention has the following advantages:

I. In the present invention, a total dilution unit is set, the dilution unit is correspondingly connected to an air extracting device, a valve group and a CEMS analyzer, and the valve group is connected to a detection device through the speed measurement device, the detection device is used to acquire the flue gas from the pipelines in different areas at the same time, so that the detection device can be centrally connected to a dilution unit, and the measurement errors of the dilution unit can be corrected and compensated by the subsequent introduction of zero gas, so as to correct the measurement precision.

II. In the present invention, a speed measurement device connected with the detection device is set, the speed measurement device is used to measure the speed of the flue gas from the pipelines in different areas, and to control the on and off of the valve group and control the working state of the air extracting device in combination with the central control unit, so that when the length of the pipeline in each area has been fixed, even if under the influence of the bending degree of the pipeline and attachments of the pipeline, the time of transporting flue gas in different pipelines to the CEMS analyzer can also be analyzed, to ensure that the flue gas of each pipeline sampled at the same time can be transported to the CEMS analyzer according to the set time sequence, to effectively eliminate real-time difference during measurement.

III. In the present invention, by setting a calibration device, the working state and precision of the speed measurement unit, the valve group, the air extracting device and the dilution unit can be calibrated and corrected only by feeding standard gas to the calibration device, thereby further ensuring the measurement reliability.

### BRIEF DESCRIPTION OF THE DRAWINGS

FIG. 1 is a structural schematic diagram of a traditional synchronous sampling and measuring system for flue gas partition at an SCR outlet;

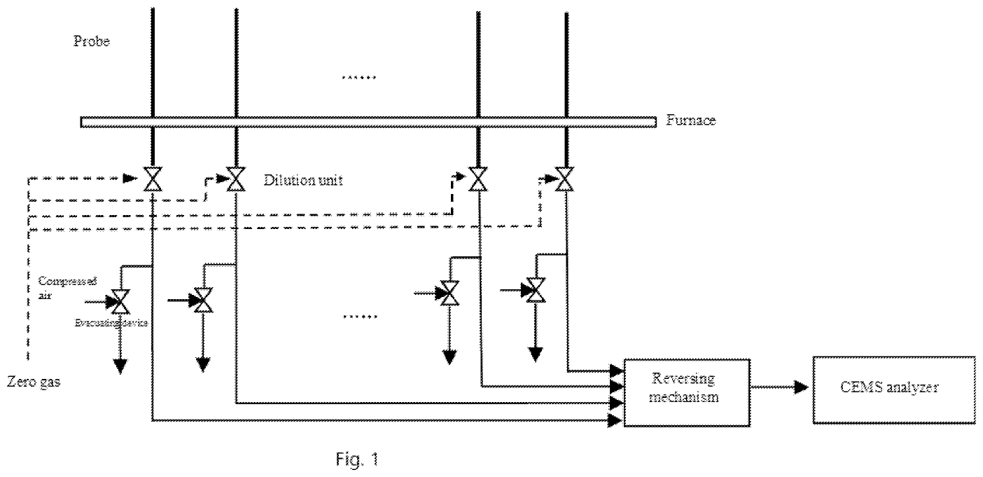

FIG. 2 is a structural schematic diagram of the present invention;

FIG. 3 is a schematic diagram of the specific working principle of the embodiment;

Reference numerals in the figures: 1, detection device, 2, speed measurement device, 3, central control unit, 4, valve group, 5, air extracting device, 6, dilution unit, 7, CEMS analyzer, 8, calibration device.

### DETAILED DESCRIPTION OF THE EMBODIMENTS

The present invention will be described in detail below in combination with accompanying drawings and a specific embodiment.

### EMBODIMENT

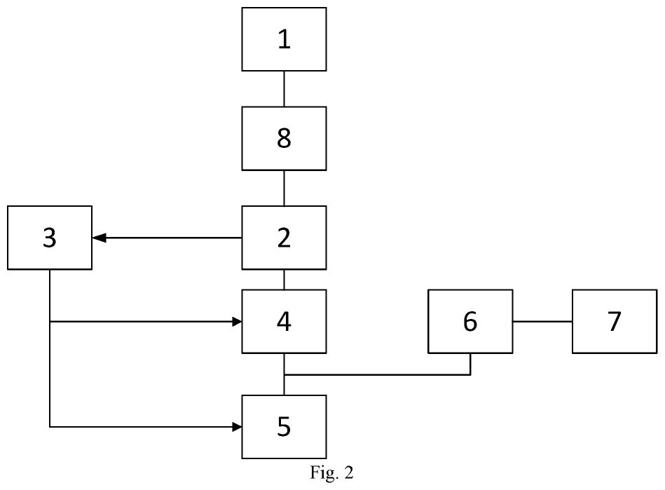

As shown in FIG. 2, a synchronous sampling and measuring system for flue gas partition includes a detection device 1 arranged at an SCR outlet for simultaneous detection of flue gas from the pipelines in different areas, wherein the detection device 1 is connected to a speed measurement device 2 for measuring the speed of flue gas, the speed measurement device 2 is connected to a central control unit 3 and one end of a valve group 4, respectively, the other end of the valve group 4 is connected to the air extracting device 5 and the dilution unit 6, respectively, the control end of the valve group 4 and the control end of the air extracting device 5 are connected to the central control unit 3, respectively, the dilution unit 6 is connected to a CEMS analyzer 7, the central control unit 3 correspondingly controls the on and off of the valve group 4 and controls the working state of the air extracting device 5 according to the data output from the speed measurement device 2, such that the flue gas sampled at the same time in the flue is transported to the air extracting device 5 according to the set time sequence. In the present embodiment, a calibration device 8 for calibrating and correcting the working state and precision of the speed measurement device 2, the valve group 4, the air extracting device 5 and the dilution unit 6 is connected between the detection device 1 and the speed measurement device 2.

When the above system is applied in practice, the specific measurement process includes the following steps:

S1, simultaneously acquiring, by the detection device 1, flue gas from pipelines in different areas at the SCR outlet;

S2, measuring, by the speed measurement device 2, the speed of the flue gas from pipelines in different areas acquired by the detection device 1, and outputting the speed measurement value of the flue gas from pipelines in each area to the central control unit 3;

S3, correspondingly controlling, by the central control unit 3, the on and off of the valve group 4 and controlling the working state of the air extracting device 5 according to the speed measurement value of the flue gas from pipelines in each area and in combination with the set control requirements, such that the flue gas sampled at the same time in different pipelines enters the air extracting device 5 according to the set time sequence;

if the valve group 4 correspondingly conducts a certain pipeline to the passage of the air extracting device 5, then the air extracting device 5 extracts the flue gas in the pipeline, so that the flue gas in the pipeline is diluted through the dilution unit 6, and then transported to the CEMS analyzer 7 for flue gas analysis, to obtain the flue gas measurement results corresponding to the pipeline;

S4, outputting, by the CEMS analyzer 7, the flue gas measurement results corresponding to pipelines in different areas, that is, the synchronous sampling and measuring process for flue gas partition is completed.

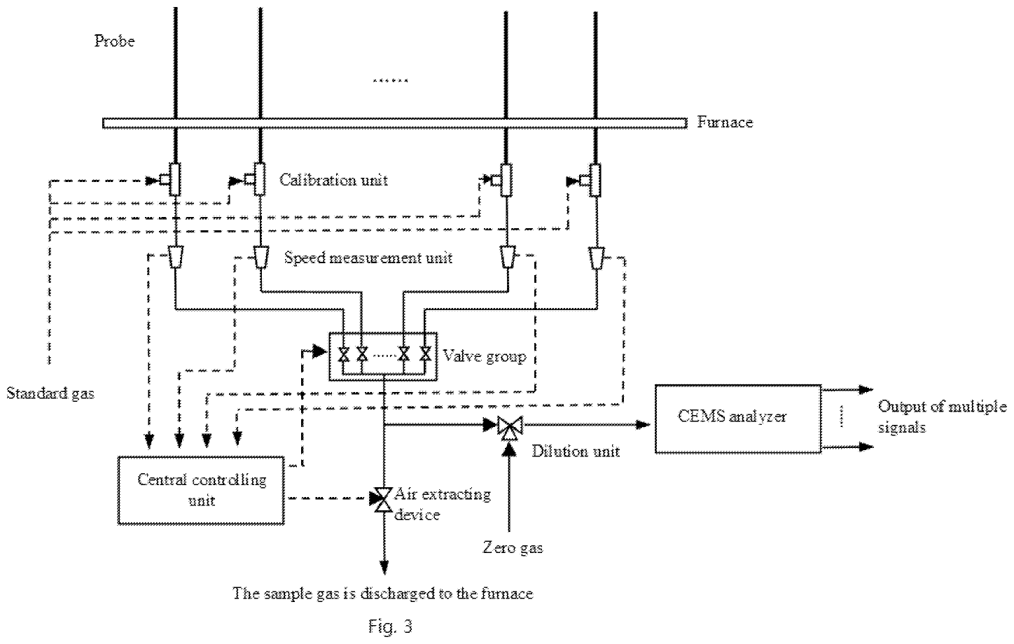

In the present embodiment, as shown in FIG. 3, the detection device 1 includes a plurality of probes to acquire the flue gas from pipelines in different areas simultaneously, and the speed measurement device 2 includes a plurality of speed measurement units corresponding to different probes to measure the speed of the flue gas from pipelines in different areas respectively, and a plurality of speed measurement units are connected to the central control unit 3 respectively, since the length of the probes and the flue gas sampling pipeline are known fixed values, so the time of flue gas from each pipeline reaching the CEMS analyzer 7 can be obtained through analysis and calculation, the central control unit 3 functions to collect the signals of the speed measurement units and control the valve group 4 and the air extracting device 5 according to the program set internally or set by the user, so that the flue gas sampled at the same time in the flue enters the air extracting device 5 according to the set time sequence;

the valve group 4 includes a plurality of valve switches corresponding to different speed measurement units, i.e., constituting a plurality of valve branches whose function is to conduct or close the passage from each sampling pipeline to the air extracting device 5, so that flue gas sampled at the same time in the flue can enter the air extracting device 5 according to the set time sequence for dilution by the dilution unit 6 and then for analysis by the CEMS analyzer 7;

the air extracting device 5 extracts the flue gas, so that the flue gas from each sampling duct can enter the CEMS analyzer 7, and the outlet of the air extracting device 5 is communicated with a furnace to discharge the extracted sample gas to the furnace;

the dilution unit 6 functions to dilute the concentration of the diluted sample gas to match the range of the CEMS analyzer 7, and the dilution unit 6 is fed with zero gas;

the CEMS analyzer 7 functions to analyze the diluted sample gas;

in addition, a plurality of calibration units are correspondingly connected between the probe and the speed measurement unit, each calibration unit is introduced with standard gas, and functions to calibrate and correct the working state and precision of the speed measurement unit, the valve group, the air extracting device and the dilution unit.

In summary, in the technical solution provided in the present invention, all the probes adopt a total dilution unit, to reduce measurement errors, since one dilution unit corresponds to a CEMS gas analyzer, the measurement errors of the dilution unit can be corrected and compensated through the introduction of zero gas, so as to correct the measurement precision.

In the present technical solution, real-time difference during measurement can be reduced through signal measurement of the speed measurement unit. When the flue gas sampling length of each pipe branch is determined unchanged, the pressure of the diluted flue gas is low and is easily affected by the bending degree of the pipeline and attachments of the pipeline, thereby easily leading to a great difference between the time at which the analyzed flue gas reaches the CEMS analyzer and the preset time, however, the time difference can be calculated in real time after the speed measurement unit measures the speed, and then the central control unit is used to control the valve group, then the real-time difference during measurement can be effectively eliminated.

We claim:

1. A synchronous sampling and measuring system for flue gas partition, comprising a detection device (1) arranged at an SCR outlet for simultaneous detection of flue gas from the pipelines in different areas, wherein the detection device (1) is connected to a speed measurement device (2) for measuring the speed of flue gas, the speed measurement device (2) is connected to a central control unit (3) and one end of a valve group (4), respectively, the other end of the valve group (4) is connected to an air extracting device (5) and a dilution unit (6), respectively, the control end of the valve group (4) and the control end of the air extracting device (5) are connected to a central control unit (3), respectively, the dilution unit (6) is connected to a CEMS analyzer (7), the central control unit (3) correspondingly controls the on and off of the valve group (4) and controls the working state of the air extracting device (5) according to the data output from the speed measurement device (2), such that the flue gas sampled at the same time in the flue is transported to the air extracting device (5) according to the set time sequence.

2. The synchronous sampling and measuring system for flue gas partition of claim 1, wherein the dilution unit (6) is fed with zero gas.

3. The synchronous sampling and measuring system for flue gas partition of claim 1, wherein an outlet of the air extracting device (5) is communicated with a furnace.

4. The flue gas partitioning synchronous sampling and measuring system for flue gas partition of claim 1, wherein a calibration device (8) for calibrating and correcting the working state and precision of the speed measurement device (2), the valve group (4), the air extracting device (5) and dilution unit (6) is connected between the detection device (1) and the speed measurement device (2).

5. The synchronous sampling and measuring system for flue gas partition of claim 4, wherein the calibration device (8) is fed with standard gas.

6. The synchronous sampling and measuring system for flue gas partition of claim 4, wherein the detection device (1) comprises a plurality of probes.

7. The synchronous sampling and measuring system for flue gas partition of claim 6, wherein the speed measurement device (2) comprises a plurality of speed measurement units corresponding to different probes.

8. The synchronous sampling and measuring system for flue gas partition of claim 7, wherein the plurality of speed measurement units are respectively connected to the central control unit (3).

9. The synchronous sampling and measuring system for flue gas partition of claim 7, wherein the valve group (4) comprises a plurality of valve switches corresponding to different speed measurement units, and the calibration device (8) comprises a plurality of calibration units corresponding to different probes.

10. Asynchronous sampling and measuring method for flue gas partition using the synchronous sampling and measuring system for flue gas partition as claimed in claim 1, comprising the following steps:
S1, simultaneously acquiring, by the detection device, flue gas from pipelines in different areas at the SCR outlet;
S2, measuring, by the speed measurement device, the speed of the flue gas from pipelines in different areas acquired by the detection device, and outputting the speed measurement value of the flue gas from pipelines in different areas to the central control unit;
S3, correspondingly controlling, by the central control unit, the on and off of the valve group and controlling the working state of the air extracting device according to the speed measurement value of the flue gas from pipelines in each area and in combination with the set control requirements, such that the flue gas sampled at the same time in different pipelines enters the air extracting device according to the set time sequence;
if the valve group correspondingly conducts a certain pipeline to the passage of the air extracting device, then the air extracting device extracts the flue gas in the pipeline, so that the flue gas in the pipeline is diluted through the dilution unit, and then transported to the CEMS analyzer for flue gas analysis, to obtain the flue gas measurement results corresponding to the pipeline; and
S4, outputting, by the CEMS analyzer, the flue gas measurement results corresponding to pipelines in different areas, that is, the synchronous sampling and measuring process for flue gas partition is completed.
